SEQUENCE LISTING (12 sequences)

SEQ ID NO: 1 - protein, 15 aa, Artificial Sequence
  Non-natural sequence
CPRLSLHRPALEDLL

SEQ ID NO: 2 - protein, 13 aa, Artificial Sequence
  Non-natural sequence
CSIPVCGQDQVTV

SEQ ID NO: 3 - protein, 14 aa, Artificial Sequence
  Non-natural sequence
CLAGLFGAAEGQAF

SEQ ID NO: 4 - protein, 13 aa, Artificial Sequence
  Non-natural sequence
CWFMPSAPYWILA

SEQ ID NO: 5 - protein, 14 aa, Artificial Sequence
  Non-natural sequence
CLTCVDLDECAIPG

SEQ ID NO: 6 - protein, 14 aa, Artificial Sequence
  Non-natural sequence
CYYVYNLTAPPECH

SEQ ID NO: 7 - protein, 14 aa, Artificial Sequence
  Non-natural sequence
CALFQTPSYTQPYQ

SEQ ID NO: 8 - protein, 13 aa, Artificial Sequence
  Non-natural sequence
CLRYMYRHKGTYH

SEQ ID NO: 9 - protein, 14 aa, Artificial Sequence
  Non-natural sequence
CEPVYVQRAKAYLE

SEQ ID NO: 10 - protein, 12 aa, Artificial Sequence
  Non-natural sequence
CRNPDEDPRGPW

SEQ ID NO: 11 - protein, 13 aa, Artificial Sequence
  Non-natural sequence
CAKQCPALEVTWP

SEQ ID NO: 12 - protein, 13 aa, Artificial Sequence
  Non-natural sequence
CVLVDPEQVVQRH

(19) United States
(12) Patent Application Publication
(10) Pub. No.: US 2019/0302115 A1
(43) Pub. Date: Oct. 3, 2019

(54) METHOD FOR REMOVING INHIBITORY COMPONENTS

(71) Applicant: Biopromic AB, Solna (SE)

(72) Inventors: Beston Hamasur, Solna (SE); Lech Ignatowicz, Sundbyberg (SE)

(73) Assignee: Biopromic AB, Solna (SE)

(21) Appl. No.: 16/336,617

(22) PCT Filed: Sep. 29, 2017
(86) PCT No.: PCT/EP2017/074812

§ 371 Date: Mar. 26, 2019

(30) Foreign Application Priority Data
Sep. 30, 2016 (US) 62402014

(51) Int. Cl. G01N 33/569; G01N 33/543

(52) U.S. Cl. CPC G01N 33/5695; G01N 33/54393; G01N 33/54313

(57) ABSTRACT

An in vitro method for detecting disease-related component(s) in a diagnostic sample including a biological fluid selected from a secreted body fluid, an excreted body fluid and cerebral spinal fluid. The method includes the steps of a) contacting the sample with a solid phase having conjugated to at least a part thereof one or more ligand(s), the ligand(s) having an affinity for and being capable of binding to one or more inhibitory component present in the sample; b) allowing the one or more inhibitory component(s) to bind to the one or more ligand(s) present on the solid phase, thereby decreasing the amount of the one or more inhibitory component(s) in the sample; and thereafter c) detecting the presence of one or more disease-related component(s) in the diagnostic sample. The one or more inhibitory component(s) are capable of binding to and interfering with the detection in step c).

### FIELD OF THE INVENTION

The present invention relates to the field of diagnostic immunoassays, and more particularly to the improvement of the sensitivity thereof.

### BACKGROUND OF THE INVENTION

Immunoassays are widely used as biochemical tests for measuring the concentration or presence of a molecule in a solution through the reaction of an antigen with an antibody. Analysis is achieved by measuring a label activity, such as radiation, fluorescence, or enzyme activity.

An important application is the diagnosis of diseases, in which an immunoassay is used for detecting small concentrations of disease-related molecules in a biological fluid. As an example, Tuberculosis (TB) is a multifaceted disease and challenging public health problem in both industrialized and developing countries, killing 3 million people a year worldwide. According to the World Health Organisation about one third of the world's population is infected with bacteria of the Mycobacterium tuberculosis complex and tuberculosis accounts for 26% of all avoidable adult death, making it the most common lethal infectious disease. Therefore, effective control of TB requires disruption of the transmission chains, which in turn requires early and accurate detection followed by immediate treatment.

Despite the enormous global burden of infectious diseases such as Tuberculosis, malaria, HIV, present tests for diagnosis of active diseases are inadequate and have severe limitations. Thus, there is a need for methods for improving the sensitivity and accuracy of diagnostic tests.

### SUMMARY OF THE INVENTION

The above presented problems have now been overcome or at least mitigated by the provision of a novel method, a solid phase and a kit comprising one or more ligand(s) capable of removing inhibitors from the bodily fluids conjugated to the solid phase.

As a first aspect of the invention, there is provided an in vitro method for detecting the presence of one or more disease-related component(s) in a diagnostic sample comprising a biological fluid selected from the group consisting of a secreted body fluid, an excreted body fluid and cerebral spinal fluid, said method comprising the steps of:
a) contacting said sample with a solid phase having conjugated to at least a part thereof one or more ligand(s), said ligand(s) having an affinity for and being capable of binding to an inhibitory component present in said sample;
b) allowing said one or more inhibitory component(s) to bind to the one or more ligand(s) present on said solid phase, thereby decreasing the amount of said one or more inhibitory component(s) in said sample; and thereafter
c) detecting the presence of one or more disease-related component(s) in said diagnostic sample,
   wherein said one or more inhibitory component(s) are characterized as capable of binding to and interfering with the detection in step c).

The present invention is based on the insight that several body fluids contains inhibitors that may block the formation of a detectable complex in an immunoassay. The inventors have thus found that the presence of inhibitory components negatively affect the test performance of immunoassays. Hence, the presence of inhibitors may result in no signal being visible or registered and hence samples may be considered as negative even if it contains the target antigen. To overcome this problem and reverse back the lost signal, the inventors have found a way to decrease the concentration of such inhibitors, which may result in a higher sensitivity in a subsequent immunoassay.

In the context of the present disclosure, the inhibitory components may comprise a mix of proteins and carbohydrates. For example, the inhibitory components may have a molecular weight of about 5-1000 kDa. In all aspects of the invention, the inhibitory component(s) may comprise at least one protein, such as at least one glycoprotein.

A preferred group of inhibitory component(s) in the context of all aspects of the present disclosure is Group 1: Ig alpha-1 chain C region, Prothrombin, Apolipoprotein D, Uromodulin, Glycophorin-C, Zinc-alpha-2-glycoprotein, Heparin sulphate proteoglycan, Phosphoinositide-3-kinase interacting protein and Interleukin 18 binding protein inhibitory component.

A further preferred group of inhibitory component(s) in the context of all aspects of the present disclosure is Group 2: Ig alpha-1 chain C region, Apolipoprotein D, Uromodulin, Glycophorin-C, Zinc-alpha-2-glycoprotein, Heparin sulphate proteoglycan, Phosphoinositide-3-kinase interacting protein, and Interleukin 18 binding protein inhibitory component.

A further preferred group of inhibitory components(s) in the context of all aspects of the present disclosure is Group 3: Apolipoprotein D, Uromodulin and Zinc-alpha-2-glycoprotein. The inventors have found that these three proteins are very potent and abundant inhibitors.

In embodiments of the first aspect of the invention, the one or more inhibitory component(s) is selected from Group 1, Group 2 or Group 3. As an example, the inhibitory components may be, or comprise, all of the inhibitory components in Group 3.

In embodiments of the first aspect of the invention, step b) further comprises the step of:
b1) separating said sample comprising said one or more disease-related component(s) from said solid phase to which said one or more inhibitory component(s) have bound.

Thus, the step of allowing said one or more inhibitory component(s) to bind to the one or more ligand(s) present on said solid phase may be followed by a step of separating the sample and the solid phase from each other. This may facilitate any future detection of a disease-related component in the sample.

Step a) involves allowing the diagnostic sample to come in contact with a solid phase onto which ligands have been conjugated. The solid phase may e.g. be a matrix or a flat surface, such as the surface of a chip or the surface of a test tube. The surface may thus be the surface of the inside of a plastic test tube. Consequently, in embodiments of the first aspect of the invention, the solid phase is a membrane or a solid surface. The step of contacting may thus involve adding the sample to a chip, injecting the sample into an analysis flow chamber in which a chip is mounted, letting the sample pass a membrane or adding the sample to a test tube.

However, the solid phase may be the surface of one or more particles, i.e. the step of contacting may be adding particles to the sample. Thus, in embodiments of the first aspect of the invention, the solid phase is the surface of one or more particle(s) having conjugated to at least a part thereof one or more of said ligand(s). The solid phase may thus comprise particles of the same kind, i.e. having the same type of ligands conjugated to the particles. However, the solid phase may comprise a first type of particle having a first type of ligand conjugated to its surface, a second type of particle having a second type of ligand conjugated to its surface, and so on.

Consequently, step a) may comprise adding to said sample one or more particle(s) having conjugated to at least a part of a surface thereof one or more ligand(s), said ligand(s) having an affinity for and being capable of binding to an inhibitory component present in said sample

Further, if particles are used, step b) may further comprise the step
b1) removing said one or more particle(s) from said biological sample

This is performed after allowing the one or more inhibitory component(s) to bind to the one or more ligand(s) present on the particle surface and may thus decrease the amount of inhibitory component in said sample.

The particle may be a particle having a surface that has been activated and onto which ligands have been immobilized. Thus, the particle may be a chemically activated particle.

For example, the particle may be a nanoparticle or a microparticle. In addition, or as an alternative, the particle may be a magnetic particle or a latex particle.

The particles may for example be removed from said biological fluid by the use of a magnet. As another example, the particles may be removed from said biological fluid by the use of centrifugation. As a further example, the particles may be removed from the biological fluid by the use of filtration.

Furthermore, step c) may comprise: contacting said sample with an anti-disease-related component antibody (detection antibody) and thereafter detecting the presence of an anti-disease-related component in said diagnostic sample. The detection antibody may be labelled in any suitable way and may for example be detected by electromagnetic spectroscopy and/or by optical density (OD) measurements. The detection may be performed by adding chemical reagent.

The detection antibody may be conjugated to a surface. Thus, step c) may for example comprise adding a particle being coated on at least a part of a surface thereof with an anti-disease-related component antibody, and thereafter detecting the presence of an anti-disease-related component in said diagnostic sample. If step a) involves contacting the sample with particles having conjugated to at least a part thereof one or more of the ligand(s), step c) may thus comprise adding a particle to said diagnostic sample, said particle being coated on at least a part of a surface thereof with an anti-disease-related component antibody, and thereafter detecting the presence of an anti-disease-related component in said diagnostic sample.

Consequently, if step a) comprises adding to said sample one or more particle(s) having conjugated to at least a part of a surface thereof one or more ligand(s), then step c) may comprise adding a second particle to said diagnostic sample, said particle being coated on at least a part of a surface thereof with an anti-disease-related component antibody, and thereafter detecting the presence of an anti-disease-related component in said diagnostic sample.

In the context of the present disclosure, the biological fluid may be selected from the group consisting of urine, sputum, saliva and cerebral spinal fluid. These are biological fluids that are well suited for a future assay using OD-measurements as detection method. Furthermore, the biological fluid may be selected from the group consisting of urine, sputum and saliva. These are biological fluids that are easy to sample, can be obtained in large quantity, and in contrast to blood they are relatively sterile and non-transmittable. Thus, such fluids are suited to use in environments where the possibilities for obtaining a sample from a patient are limited. As an example, the biological fluid may be urine.

The disease-related component(s) may comprise an antigen, i.e. a molecule capable of inducing an immune response in an organism, or a metabolite thereof. For example, the disease-related component(s) may comprise an exogenous antigen, i.e. an antigen that has entered the body from the outside by means of e.g. injection or inhalation. However, the antigen may be an endogenous antigen or a tumor antigen

In embodiments of the first aspect of the invention, the one or more disease-related component(s) comprises at least one polysaccharide.

The one or more disease-related components may be of human or pathogen origin. The one or more disease-related component(s)s may comprise whole bacteria, cells, virus or may be fragments thereof, such as cell wall components. Further, the one or more disease-related components may comprise proteins, carbohydrates or be degradation products from proteins or carbohydrates.

In embodiments of the first aspect, the one or more disease-related component(s) comprises at least one pathogen-derived component. The pathogen-derived component may be a polysaccharide. A pathogen is an infectious agent such as a virus, bacterium, protozoa, prion, fungus or other microorganisms that may invade a host organism. A pathogen-derived component is thus a molecule that originate or stems from such a pathogen. The pathogen-derived component may further be detected in all types of biological samples, including blood. Thus, in an alternative aspect of the present invention, there is provided an in vitro method for detecting the presence of one or more pathogen-derived component(s) in a diagnostic sample comprising a biological fluid, said method comprising the steps of:
a) contacting said sample with a solid phase having conjugated to at least a part thereof one or more ligand(s), said ligand(s) having an affinity for and being capable of binding to an inhibitory component present in said sample;
b) allowing said one or more inhibitory component(s) to bind to the one or more ligand(s) present on said solid phase, thereby decreasing the amount of said one or more inhibitory component(s) in said sample; and thereafter
c) detecting the presence of one or more disease-related component(s) in said diagnostic sample,
   wherein said one or more inhibitory component(s) are characterized as capable of binding to and interfering with the detection in step c).

The biological fluid may be selected from the group consisting of blood, urine, sputum, saliva and cerebral spinal fluid.

As an example, the disease-related component may be a Mycobacterium Tuberculosis antigen, such as LAM, or a metabolite thereof. LAM is the major Mycobacterium tuberculosis surface antigen lipoarabinomannan. A metabolite of LAM may be LAM degradation fragments, such as delipidated LAM.

Further examples of disease-related components include Phosphoinositol mannoside, Lipomannan, C-polysaccharide S. pneumoniae, and PC (Phosphocholine)-human endogenous antigen, an intermediate in synthesis of phosphatidylcholine.

As discussed above, in all aspects of the present invention, the ligand used may have an affinity for a protein, such as a glycoprotein. The protein may be any one of the inhibitory components listed in Group 1, Group 2 or Group 3 above.

The ligands may thus have affinity for at least one inhibitory component in Group 1, Group 2 or Group 3. The ligands conjugated to the solid phase may thus have affinity for and being capable of binding to any number of inhibitory components in Group 1, Group 2 or Group 3. As an example, the ligands conjugated to the solid phase may have affinity for and being capable of binding to all inhibitory components in Group 1, Group 2 or Group 3.

In embodiments of the first aspect of the invention, the ligands conjugated to the solid phase have affinity for at least two inhibitory components in Group 1 or Group 2, such as affinity for at least three, such as at least five inhibitory components in Group 1 or Group 2.

In embodiments of the first aspect of the invention, the ligands conjugated to the solid phase have affinity for at least one, such as for at least two, such as for all inhibitory components in Group 3.

In embodiments of the first aspect of the invention, the ligands conjugated to the solid phase have affinity for at least one, such as for at least two, such as for all inhibitory components of Group 3, and affinity for at least one, such as at least two, such as at least three, such as at least four, such as at least five inhibitory components selected from the group consisting of Ig alpha-1 chain C region, Prothrombin, Glycophorin-C, Heparin sulphate proteoglycan, Phosphoinositide-3-kinase interacting protein and Interleukin 18 binding protein inhibitory component.

In embodiments of the first aspect of the invention, the ligands conjugated to the solid phase have affinity for all inhibitory components of Group 3, and affinity for at least one, such as at least two, such as at least three, such as at least four, such as at least five inhibitory components selected from the group consisting of Ig alpha-1 chain C region, Prothrombin, Glycophorin-C, Heparin sulphate proteoglycan, Phosphoinositide-3-kinase interacting protein and Interleukin 18 binding protein inhibitory component.

In embodiments of the first aspect of the invention, the ligands conjugated to the solid phase have affinity for at least one, such as for at least two, such as for all inhibitory components of Group 3, and affinity for at least one, such as at least two, such as at least three, such as at least four, such as at least five inhibitory components selected from the group consisting of Ig alpha-1 chain C region, Glycophorin-C, Heparin sulphate proteoglycan, Phosphoinositide-3-kinase interacting protein and Interleukin 18 binding protein inhibitory component.

In embodiments of the first aspect of the invention, the ligands conjugated to the solid phase have affinity for all inhibitory components of Group 3, and affinity for at least one, such as at least two, such as at least three, such as at least four, such as at least five inhibitory components selected from the group consisting of Ig alpha-1 chain C region, Glycophorin-C, Heparin sulphate proteoglycan, Phosphoinositide-3-kinase interacting protein and Interleukin 18 binding protein inhibitory component.

The solid phase may thus comprise different ligands having affinity for different inhibitory components, such as the inhibitory components listed in Group 1, Group 2 or Group 3 above. As an example, at least two, such as at least three, such as at least five different types of ligands may be conjugated to the solid phase, wherein each type has affinity for a different inhibitory component.

The ligand may be a biological molecule, such as a synthetic peptide(s). As an example, the ligand may be a biological molecule that has an affinity for one of more of the inhibitory components listed in Group 1, Group 2 or Group 3 above.

Furthermore, the biological molecule may be a peptide comprising an amino sequence selected from Group 4:

(SEQ ID NO: 1)
CPRLSLH RPALEDLL
(SEQ ID NO: 2)
CSIPVCGQDQ VTV
(SEQ ID NO: 3)
CLAGLFGAAEG QAF
(SEQ ID NO: 4)
CWFMPSAPYWI LA
(SEQ ID NO: 5)
CLTCVDLDECA IPG
(SEQ ID NO: 6)
CYYVYNLTAPP ECH
(SEQ ID NO: 7)
CALFQTPSYTQ PYQ
(SEQ ID NO: 8)
CLRYMYRHKGT YH
(SEQ ID NO: 9)
CEPVYVQRAKA YLE
(SEQ ID NO: 10)
C RNPDEDPRGP W
(SEQ ID NO: 11)
C AKQCPALEVTWP
(SEQ ID NO: 12)
C VLVDPEQVVQRH

The peptides comprising an amino sequence selected from Group 4 may have affinity for at least one, such as all, inhibitory components of Group 1 or Group 2.

One or several of the above peptides, such as at least two, such as at least three, such as at least five of the above peptides, may be conjugated to the solid phase. As an example, all different types of peptides above may be conjugated to the solid phase, such as to a surface or a single particle. As an example, the solid phase may comprise particles having more than one or all of the peptides randomly conjugated to the particles to give a more or less equal distribution of peptides among the particles. As an alternative, the solid phase may comprise a first type of particle having only one or a few of the peptides conjugated to the surface as well as a second type of particle having other types of peptides conjugated to the surface, and so on. As an example, the solid phase may comprise a number of different types of particles, each type having a different type of ligand conjugated to it surface as compared to the other types.

Furthermore, the ligand may be a chemical molecule. As an example, the ligand may be selected from the group consisting of: 4-mercaptophenylboronic acid, amine benzenediazonium compounds, and Polymixine. Several chemical molecules in the above group may simultaneously be used as ligands.

As a configuration of the first aspect of the invention, there is provided an in vitro method for removing one or more inhibitory component(s) from a diagnostic sample comprising a biological fluid, said method comprising the steps of:
a) to said sample, adding one or more particle(s) having conjugated to at least a part of a surface thereof one or more ligand(s), said ligand(s) having an affinity for and being capable of binding to an inhibitory component present in said sample;
b) allowing said one or more inhibitory components to bind to said particles; and
c) removing said particles from the sample,
   wherein said inhibitory component(s) is/are characterized as components capable of interfering with said diagnostic sample when used in a subsequent immunoassay.

The biological fluid may be selected from the group consisting of a secreted body fluid, an excreted body fluid and cerebral spinal fluid as discussed herein above.

As a further configuration of the first aspect of the invention, there is provided an in vitro method for preparing and/or cleaning a diagnostic sample comprising a biological fluid for a subsequent immunoassay, said method comprising the steps of:
a) to said sample, adding one or more particle(s) having conjugated to at least a part of a surface thereof one or more ligand(s), said ligand(s) having an affinity for and being capable of binding to an inhibitory component present in said sample;
b) allowing said one or more inhibitory components present in said sample to bind to said one or more ligand(s) present on said particles; and thereafter
c) obtaining a cleaned and/or prepared diagnostic sample prepared for an immunoassay,
   wherein said inhibitory components are characterized as capable of binding to the assay components and also interfering with a diagnostic immunoassay.

The biological fluid may be selected from the group consisting of a secreted body fluid, an excreted body fluid and cerebral spinal fluid as discussed herein above.

As a second aspect of the invention, there is provided an in vitro method for detecting the presence of one or more disease-related component(s) in a diagnostic sample comprising a biological fluid selected from the group consisting of a secreted body fluid, an excreted body fluid and cerebral spinal fluid, said method comprising the steps of:
a) decreasing the amount of one or more inhibitory components in said biological fluid to provide a cleaned diagnostic sample, wherein said one or more inhibitory component(s) comprises at least one protein; and
b) detecting the presence of said one or more disease-related component(s) in the cleaned diagnostic sample of step a).

The terms and definitions used in relation to the second aspect are as discussed for the first aspect above

The inhibitory components may comprise at least one protein, such as at least one glycoprotein. In embodiments of the second aspect of the invention, one or more inhibitory component(s) is selected from Group 1, Group 2 or Group 3 of inhibitory components disclosed herein above.

Step a) may thus involve decreasing the amount of any number of inhibitory components in Group 1 Group 2 or Group 3, such as decreasing all inhibitory components in Group 1, Group 2 or Group 3.

In embodiments of the second aspect of the invention, step a) comprises decreasing the amount of at least two inhibitory components in Group 1, Group 2 or Group 3, such as decreasing the amount of at least three, such as at least five inhibitory components in Group 1 or Group 2.

In embodiments of the second aspect of the invention, step a) comprises decreasing the amount of at least one, such as at least two, such as all inhibitory components of Group 3, and decreasing the amount of at least one, such as at least two, such as at least three, such as at least four, such as at least five inhibitory components selected from the group consisting of Ig alpha-1 chain C region, Prothrombin, Glycophorin-C, Heparin sulphate proteoglycan, Phosphoinositide-3-kinase interacting protein and Interleukin 18 binding protein inhibitory component.

In embodiments of the second aspect of the invention, step a) comprises decreasing the amount of at least one, such as at least two, such as all inhibitory components of Group 3, and decreasing the amount of at least one, such as at least two, such as at least three, such as at least four, such as at least five inhibitory components selected from the group consisting of Ig alpha-1 chain C region, Glycophorin-C, Heparin sulphate proteoglycan, Phosphoinositide-3-kinase interacting protein and Interleukin 18 binding protein inhibitory component.

As a third aspect of the invention, there is provided a solid phase for use in the removal of inhibitory components from a diagnostic sample before subsequent use of said diagnostic sample in an immunoassay, said solid phase having one or more ligand(s) conjugated to at least a part thereof, said ligand(s) having an affinity for and being capable of binding to an inhibitory component, wherein said inhibitory component is characterized as capable of interfering with said diagnostic immunoassay.

The terms and definitions used in relation to the third aspect are as discussed for the first aspect above. As an example, the solid phase may have at least two ligands conjugated to at least a part thereof. Thus, the third aspect may provide a solid phase for use in the removal of inhibitory components from a diagnostic sample before subsequent use of said diagnostic sample in an immunoassay, said solid phase having at least two different types ligands conjugated to at least a part thereof, said ligands having an affinity for and being capable of binding to different inhibitory components, wherein said inhibitory components are characterized as capable of interfering with said diagnostic immunoassay.

For example, the ligand or ligands may be as defined in any embodiment of the first aspect above.

In embodiments of the third aspect of the invention, the solid phase has different ligands conjugated to at least part thereof, so that said ligands have affinity for at least two inhibitory components selected from Group 1, Group 2 or Group 3 herein above.

As an example, the solid phase may be at least one particle. Further, the particle may be a nanoparticle or a microparticle. As a further example, the particle may be a magnetic particle or a latex particle.

The surface of the particle may also have been activated in order to bind ligands to the surface. Thus, the particle may be a surface activated magnetic particle.

As a fourth aspect of the invention, there is provided the use of a solid phase according to the third aspect of the invention for preparing and/or cleaning a diagnostic sample for use in a subsequent immunoassay. The terms and definitions used in relation to the fourth aspect are as discussed for the other aspects above. The use may involve removing or decreasing the amount of one or more inhibitory component(s) from said diagnostic sample before a diagnostic immunoassay is performed. The inhibitory components may be selected from Group 1, Group 2 or Group 3 herein above. The use may thus involve decreasing the amount of any number of inhibitory components in Group 1, Group 2, or Group 3, such as decreasing all inhibitory components in Group 1, Group 2 or Group 3.

Furthermore, the use may comprise decreasing the amount of at least two inhibitory components in Group 1, Group 2 or Group 3, such as decreasing the amount of at least three, such as at least five inhibitory components in Group 1, Group 2 or Group 3.

In embodiments of the fourth aspect of the invention, the use comprises decreasing the amount of at least one, such as at least two, such as all inhibitory components of Group 3, and decreasing the amount of at least one, such as at least two, such as at least three, such as at least four, such as at least five inhibitory components selected from the group consisting of Ig alpha-1 chain C region, Prothrombin, Glycophorin-C, Heparin sulphate proteoglycan, Phosphoinositide-3-kinase interacting protein and Interleukin 18 binding protein inhibitory component.

In embodiments of the fourth aspect of the invention, the use comprises decreasing the amount of at least one, such as at least two, such as all inhibitory components of Group 3, and decreasing the amount of at least one, such as at least two, such as at least three, such as at least four, such as at least five inhibitory components selected from the group consisting of Ig alpha-1 chain C region, Glycophorin-C, Heparin sulphate proteoglycan, Phosphoinositide-3-kinase interacting protein and Interleukin 18 binding protein inhibitory component.

As a fifth aspect of the invention, there is provided a kit of parts comprising:
a) means for capturing and detecting one or more disease-related components in an immunoassay, and
b) means for decreasing the amount of one or more inhibitory components in a biological fluid, wherein said one or more inhibitory component(s) comprises at least one protein.

In embodiments of the fifth aspect of the invention, the means for decreasing the amount of one or more inhibitory components in a biological fluid comprises one or more solid phase as according to the third aspect above. Thus, the kit may comprise particles onto which ligands against inhibitory components as disclosed herein have been conjugated. However, the means for decreasing the amount of one or more inhibitory components in a biological fluid may comprise a solid phase, such as a membrane, a matrix, a chip surface or the inner surface of a test tube, onto which ligands against the one or more inhibitory components have been immobilized.

The at least one or more inhibitory component(s) comprises at least one protein, such as at least one glycoprotein. The inhibitory components may be selected from the inhibitory components listed in Group 1, Group 2 or Group 3 above.

Further, the means for decreasing the amount of one or more inhibitory components mat comprise means for decreasing at least two different inhibitory components, such as at least two inhibitory components selected from Group 1, Group 2 or Group 3 above.

The means for capturing and detecting one or more of the disease-related components in an immunoassay may comprise an anti-disease-related component antibody (detection antibody), such as an anti-disease-related component antibody conjugated to particles other than the particles onto which ligands against inhibitory components have been conjugated.

Furthermore, in embodiments of the fifth aspect of the invention, the one or more disease-related components is a Mycobacterium Tuberculosis antigen, such as LAM.

### BRIEF DESCRIPTION OF THE DRAWINGS

FIG. 1 shows a general method of the present disclosure for cleaning a specimen before an immunoassay. (A) with use of ligands: on particles (B), solid surface (C), filter matrix (D).

FIG. 2 shows the SpeClean (Specimen Cleaner) improvement of LAM-Tb assay in various body fluids (100 pg/ml).

FIG. 3 shows the Effect of SpeClean on the improvement of assay sensitivity in urine samples spiked with various antigens (100 pg/ml).

FIG. 4 demonstrates the difference in clinical sensitivity for an immune assay (detection of LAM in urine) when urine sample is pretreated with the SpeClean. Sensitivity untreated=47.6%, SpeClean treated=80.9%, n=21, LOD 0.4 OD 620 nm.

### DETAILED DESCRIPTION

The inventors have found a way to decrease the amount of inhibitory compounds in biological samples so that the sensitivity in a subsequent immunoassay is increased.

For example, if the major Mycobacterium tuberculosis surface antigen lipoarabinomannan (LAM) is added to urine from healthy individuals or Tuberculosis (TB) patients the LAM signal is frequently reduced or even totally quenched, when compared with the same amount of LAM in PBS or synthetic urine. This inhibitory effect varies between individuals, and also between samples taken at various time points from the same individual.

Previous proteomic studies have identified more than 2800 urine proteins from healthy individual, however their impact in immunoassay haven't yet been explored. Using various chromatographic methods in combination with mass spectrometric analysis, the inventors have for example now been able to isolate, purify and characterized several abundant proteins from healthy urine with potential inhibitory effect on LAM immunoassay.

When reconstituted in LAM spiked phosphate buffer saline or synthetic urine imitation, these proteins alone or in combination exert inhibitory effect on the test either by masking LAM antigen or by interaction with the detector antibody. Reducing the inhibitory effects of patient urines obviously would increase the diagnostic sensitivity and accuracy of a LAM based urine test.

There is presented herein the inhibitory effect of these proteins on an immunoassay, an understanding of the mechanism of action and also finding the way to remove them from the body fluid and hence restoring sensitivity of the assay. This provides great advantages for the sensitivity of an immunoassay. Herein, the LAM assay is used as an example of an immunoassay that can benefit from pre-cleaning of the diagnostic sample, but this process is also applicable to other immunoassays.

Urine is a good matrix to be used for diagnosis of various diseases as it is sterile, can be obtained in larger volume compare to blood or any other fluid and most importantly no needle is needed as in the case of blood and hence avoiding further intra infections with for example hepatitis and HIV.

To that end, two approaches to construct specimen cleaners (SpeClean) for the removal of the inhibitors from the body fluid were used:
A) Identification of peptides with binding affinity toward the urinary inhibitors by phage display technology. Twelve peptides acting as ligands toward the inhibitory proteins listed below were identified and conjugated to magnetic particle (MP) for easy removal of the inhibitors from the body fluid by magnet.
B) Our understanding on the structural differences between the target glycolipid antigen (LAM) and the carbohydrate moiety of the inhibitory proteins (mainly consisting of N-acetylglucosamine) helped to identify several ligands with binding capacity for the inhibitors. These ligands were then used to construct many ligand-MP conjugates for simple removal of the inhibitors from the body fluids.

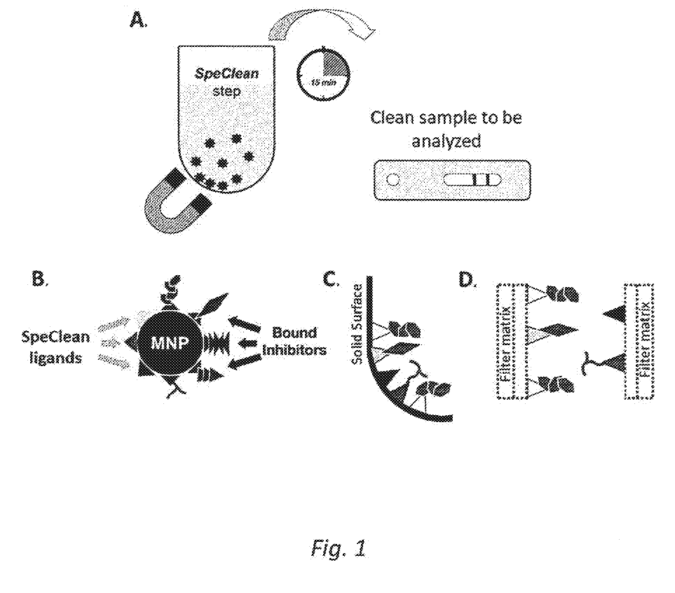

Hence, there is provided herein for the first time a method for removal of inhibitors from a biological sample (body fluid) comprising urine or other bodily fluids, such as plasma, sputum, saliva etc., before said biological sample is used in a subsequent diagnostic assay, such as an immunoassay. To remove said inhibitors, a particle, such as a nanoparticle or a micro particle may be used herein. Said particle may e.g. be a latex or a magnetic particle. The general method is illustrated in FIG. 1. A) shows how magnetic particles onto which ligands have been immobilized may be removed from a sample using magnetism after incubation, i.e. after having bound inhibitory components, B) shows ligands (SpeClean ligands) immobilized onto a magnetic particle (MNP) and with bound inhibitors, C) shows ligands bound to a solid surface and D) shows ligands bound to a filter matrix.

There are many manufacturers offering various kinds of particles, including magnetic particles with diverse surfaces, sizes and functionality that may be used in the current context.

The specimen cleaner (e.g. a particle comprising the ligands (chemical or biological) conjugated to at least a part of a surface thereof as defined herein) is a technique that may be used for removing inhibitors from any biological fluids. The approach can be used in conjunction to varieties of diagnostic methods and is hence not limited to LAM assay. Conjugation of the ligands to the particle may be performed by conventional means.

Examples of Inhibitory Proteins and Ligands

In the below is a list of isolated proteins (inhibitory proteins interfering with a diagnostic assay), forming the basis for the identification of biological molecules binding thereto for use as ligands in the cleaning and/or preparation of diagnostic samples for subsequent use in an immunoassay. There is also provided a list of chemical ligands presenting examples for use as ligands in a method as disclosed herein.

Example of Proteins with Inhibitory Effect in an Immunoassay (Group 1)

Ig alpha-1 chain C region

Prothrombin

Apolipoprotein D

Uromodulin

Glycophorin-C

Zinc-alpha-2-glycoprotein

Heparin sulphate proteoglycan

Phosphoinositide-3-kinase interacting protein

Interleukin 18 binding protein

Peptide Ligands (Group 4)

(SEQ ID NO: 1)
CPRLSLH RPALEDLL
(SEQ ID NO: 2)
CSIPVCGQDQ VTV
(SEQ ID NO: 3)
CLAGLFGAAEG QAF
(SEQ ID NO: 4)
CWFMPSAPYWI LA
(SEQ ID NO: 5)
CLTCVDLDECA IPG
(SEQ ID NO: 6)
CYYVYNLTAPP ECH
(SEQ ID NO: 7)
CALFQTPSYTQ PYQ
(SEQ ID NO: 8)
CLRYMYRHKGT YH
(SEQ ID NO: 9)
CEPVYVQRAKA YLE
(SEQ ID NO: 10)
CR NPDEDPRGP W
(SEQ ID NO: 11)
C AKQCPALEVTWP
(SEQ ID NO: 12)
C VLVDPEQVVQRH

Chemical Ligands (Examples)

4-mercaptophenylboronic acid

Amine benzenediazonium compounds

Polymixine

### EXPERIMENTAL SECTION

The experimental section illustrates the positive effects of using a cleaning step removing any inhibitors from the diagnostic sample on the antigen, e.g. (LAM) detection.

Preparation of the Specimen Cleaner (SpeClean)

Conjugation of the Peptides (Anti Inhibitor) to Various Matrixes

Magnetic micro-particles, nitrocellulose membrane, latex beads and frits, all functionalized with amino groups were employed for the immobilization/conjugation of the peptide ligands. An equal amount of all peptides in Group 4 were conjugated to solid phases. The amino groups were first converted to bromine through bromoalkylation reaction with Bromoacetic acid N-hydroxysuccinimide ester. Next the cysteinylated peptides were added dropwise to the activated matrix while maintaining the pH. To maximize the conjugation yield, tributylphosphine at 10 mM was added to the reaction mixture to keep the thiol groups from oxidation and hence allowing for the reaction with bromine on the matrix surfaces.

The resulting specimen cleaner was denoted SpeClean 1. This may be added to a sample and after incubation be removed so as to decrease the amount of inhibitory compounds. For example, the magnetic particles can be removed by a magnet.

Conjugation of Chemical Ligands to Matrix Surfaces

Conjugation of the 4-mercaptophenylboronic acid to the matrix surface was performed as described above. For the immobilization of benzenediazonium compounds and polymixin, carboxyl (COOH) functionalized matrixes were used. Briefly, the matrix first equilibrated with 2-(N-morpholino) ethanesulfonic acid at pH 6.0 followed by activation with, N-hydroxysuccinimide (NHS) and 1-Ethyl-3-(3-dimethylaminopropyl)-carbodiimide (EDC) for 1 hour at room temperature. After washing the amine containing ligands were added and the conjugation allowed to occur for 2 hours at room temperature.

The resulting specimen cleaner was denoted SpeClean 2.

General Assay Description (LAM Assay, Detecting LAM Antigen)
Magnetic particles coated with capture anti LAM antibody are added to urine/other body fluid and incubated
After washing, biotinylated detector antibody is added and incubated.
After washing avidin-HRP enzyme conjugate is added and incubated.
After washing tetramethylbenzidine, TMB substrate is added and the intensity of the developed color (optical density registered at 450 nm).

Since the body fluid may contains inhibitors that block the formation of any color a lower/no signal will be visible/registered and hence the sample will be considered as negative even if it contains the target antigen. To overcome this problem and reverse back the lost signal, the fluid needs to be cleaned up from the inhibitors. To that end, the specimen cleaning step of the present disclosure is applied using SpeClean 1 or SpeClean 2 for the cleaning of the inhibitor prior to the addition of the capture particles. Hence, a cleaning step is introduced before the assay/method described in the above is performed.

### Results

### Example. 1

In the below, example 1 is a LAM (antigen) assay, and examples 2 to 4 comprise the additional specimen cleaner step being performed before antigen (LAM) assay is performed. The data show that performing a cleaning step prior to the antigen detection test (in this context, LAM) improves the sensitivity of the assay as any inhibitory molecules present in the sample and binding to the antigen are removed before capture particles binding to antigen (LAM) are added to the biological sample.

| | Signal at 450 nm |
|---|---|
| Synthetic urine | 0.251 |
| Synthetic urine + 100 pg/ml LAM | 4.440 |
| Urine | 0.28 |
| Urine + 100 pg/ml LAM | 0.31 |

In synthetic urine spiked with LAM, the signal for LAM is higher due to the non-presence of inhibitory molecules therein. The signal for LAM however, is low or almost non-existing in urine spiked with LAM.

### Example 2

Effect of SpeClean on the Urine of 1 Healthy Donor Spiked with +/−100 pg/ml LAM

| Samples | SpeClean | OD 450 nm |
|---|---|---|
| Urine | − | 0.14 |
| Urine + 100 pg LAM/ml | − | 0.17 |
| Urine + 250 pg LAM/ml | − | 0.51 |
| Urine + 500 pg LAM/ml | − | 0.93 |
| Urine | + | 0.22 |
| Urine + 100 pg LAM/ml | + | 3.11 |
| Urine + 250 pg LAM/ml | + | 4.12 |
| Urine + 500 pg LAM/ml | + | 7.74 |

This illustrates that a SpeClean step (i.e. removing the inhibitors in the sample) prior to performing the assay increases the sensitivity of antigen (LAM) detection test. A low/no signal for antigen (LAM) is obtained if no cleaning step is performed.

### Example 3

Effect of SpeClean on the urine of 5 healthy donors with various degree of inhibition. In this experiment the urine samples were spiked with 100 pg/ml LAM.

| Samples | SpeClean | 450 nm |
|---|---|---|
| D1 | − | 0.400 |
| D1 | + | 4.149 |
| D2 | − | 3.88 |
| D2 | + | 4.30 |
| D3 | − | 1.34 |
| D3 | + | 4.19 |
| D4 | − | 3.65 |
| D4 | + | 4.40 |
| D5 | − | 0.31 |
| D5 | + | 2.88 |

| | |
|---|---|
| Synthetic urine (no LAM) | 0.251 |
| Synthetic urine + 100 pg/ml LAM | 4.440 |

This experiment clearly illustrates the presence of various amount of inhibitors in individual urine ranging from low (D2, D4), medium (D3) and high (D1, D5) that revers back to the normal signal (LAM-spiked synthetic urine) after treatment with specimen cleaner.

### Example 4

Effect of Two Different SpeClean Compositions, SpeClean 1 and SpeClean 2 (See Above,) on Healthy Urine Spiked with LAM.

| Samples | SpeClean | 450 nm |
|---|---|---|
| 1- Urine | — | 0.22 |
| 2- Urine + 100 pg/ml | — | 0.24 |
| 3- Urine | 1 | 0.18 |
| 4- Urine + 100 pg/ml | 1 | 4.22 |
| 5- Urine + 50 pg/ml | 1 | 2.88 |
| 6- Urine + 20 pg/ml | 1 | 2.11 |
| 7- Urine | 2 | 0.28 |
| 8- Urine + 100 pg/ml | 2 | 4.78 |
| 9- Urine + 50 pg/ml | 2 | 3.11 |
| 10- Urine + 20 pg/ml | 2 | 2.25 |

This experiment shows that the specimen cleaner based on biological ligand (SpeClean 1) performs as good as the chemical-based specimen cleaner (SpeClean 2).

### Example 5

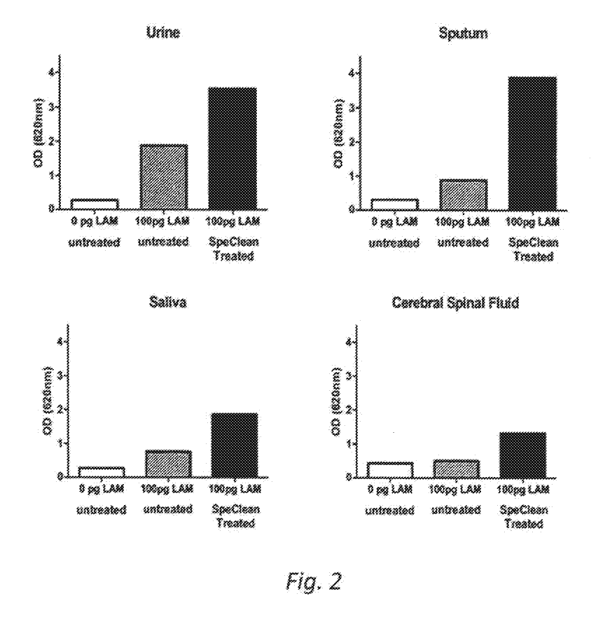

Experiments were performed to see the effect of the SpeClean improvement of a LAM-TB assay in various body fluids. Urine, sputum, saliva and cerebral spinal fluid with and without addition of SpeClean 1 were tested in an assay as described in in Examples 1-4 above. The results are shown in FIG. 2, which clearly shows that addition of the SpeClean 1 composition clearly enhances the OD for in the assay of all biological samples.

### Example 6

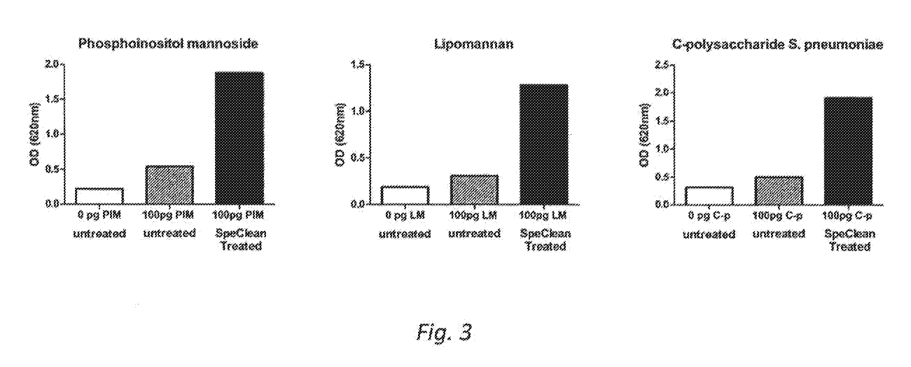

Experiments were performed to see the effect of the SpeClean improvement when performing an assay in urine samples with addition of various antigens: phosphoinositol mannoside, lipomannan and C-polysaccharide S. pneumoniae. The results are shown in FIG. 3 and it is evident that all samples treated with SpeClean1 clearly improved the OD at 620 nm compared to the untreated samples, i.e. the assay sensitivity was increased for all antigens tested.

### Example 7

The effect of the SpeClean improvement of assay sensitivity was tested in urine samples collected from 21 different TB patients with confirmed pulmonary tuberculosis.

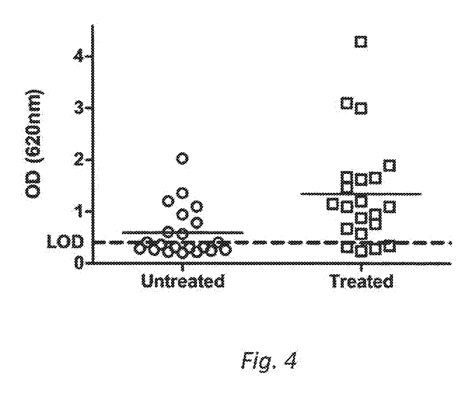

FIG. 4 demonstrates the difference in clinical sensitivity for an immune assay (detection of LAM in urine) when urine sample is pretreated with the SpeClean 1 composition. Pre-treatment with SpeClean 1 results in increased positive signal in majority of the samples and results in overall improvement of clinical sensitivity of the assay from identifying correctly 31.8% of infected individuals to 80.9% infected patients.

### Example 8

Peptide ligands against Apolipoprotein D, Uromodulin and Zinc-alpha-2-glycoprotein are conjugated to magnetic microparticles according to the conjugation scheme disclosed herein above to give a Specimen cleaner denoted SpeClean 3.

SpeClean 3 is added to a urine sample and also removed after incubation before antigen (LAM) assay is performed according to the general assay description above.

### Example 9

Peptide ligands against Apolipoprotein D, Uromodulin and Zinc-alpha-2-glycoprotein are conjugated to a solid surface according to the conjugation scheme disclosed herein above to give a Specimen cleaner denoted SpeClean 4.

A urine sample is contacted with the SpeClean 4 surface and left to incubate. The sample is then separated from the surface and before antigen (LAM) assay is performed according to the general assay description above.

### Example 10

Peptide ligands against Apolipoprotein D, Uromodulin and Zinc-alpha-2-glycoprotein are conjugated to the inside surface of a test tube according to the conjugation scheme disclosed herein above to give a Specimen cleaner denoted SpeClean 5.

A urine sample is added to the test tube and left to incubate before being removed from the test tube. An antigen (LAM) assay of the sample is then performed according to the general assay description above.

1-48. (canceled)

49. An in vitro method for detecting the presence of one or more disease-related component(s) in a diagnostic sample comprising a biological fluid selected from the group consisting of a secreted body fluid, an excreted body fluid and cerebral spinal fluid, said method comprising the steps of:
a) contacting said sample with a solid phase having conjugated to at least a part thereof one or more ligand(s), said ligand(s) having an affinity for and being capable of binding to one or more inhibitory component present in said sample;
b) allowing said one or more inhibitory component(s) to bind to the one or more ligand(s) present on said solid phase, thereby decreasing the amount of said one or more inhibitory component(s) in said sample; and thereafter
c) detecting the presence of one or more disease-related component(s) in said diagnostic sample,
wherein said one or more inhibitory component(s) are characterized as capable of binding to and interfering with the detection in step c) and selected from the group consisting of Ig alpha-1 chain C region, Prothrombin, Apolipoprotein D, Uromodulin, Glycophorin-C, Zinc-alpha-2-glycoprotein, Heparin sulphate proteoglycan, Phosphoinositide-3-kinase interacting protein, and Interleukin 18 binding protein inhibitory component.

50. The in vitro method of claim 49, wherein step b) further comprises the step of:
b1) separating said sample comprising said one or more disease-related component(s) from said solid phase to which said one or more inhibitory component(s) have bound.

51. The in vitro method of claim 49, wherein said solid phase is the surface of one or more particle(s) having conjugated to at least a part thereof one or more of said ligand(s).

52. The in vitro method of claim 49, wherein step c) comprises: contacting said sample with an anti-disease-related component antibody (detection antibody) and thereafter detecting the presence of an anti-disease-related component in said diagnostic sample.

53. The in vitro method of claim 52, wherein step c) comprises: adding a particle to said diagnostic sample, said particle being coated on at least a part of a surface thereof with an anti-disease-related component antibody, and thereafter detecting the presence of an anti-disease-related component in said diagnostic sample.

54. The in vitro method of claim 49, wherein said biological fluid is selected from the group consisting of urine, sputum, saliva and cerebral spinal fluid.

55. The in vitro method of claim 49, wherein said one or more disease-related component(s) comprises at least one pathogen-derived component.

56. The in vitro method of claim 55, wherein said disease-related component is a Mycobacterium tuberculosis antigen.

57. The in vitro method of claim 55, wherein said disease-related component is selected from Phosphoinositol mannoside, Lipomannan, C-polysaccharide S. pneumoniae and PC (Phosphocholine)-human endogenous antigen.

58. The in vitro method of claim 49, wherein said ligand(s) is a biological molecule.

59. The in vitro method of claim 49, wherein said solid phase comprises different ligands having affinity for different inhibitory components.

60. An in vitro method for detecting the presence of one or more disease-related component(s) in a diagnostic sample comprising a biological fluid selected from the group consisting of a secreted body fluid, an excreted body fluid and cerebral spinal fluid, said method comprising the steps of:
a) decreasing the amount of one or more inhibitory components in said biological fluid to provide a cleaned diagnostic sample, wherein said one or more inhibitory component(s) comprises are selected from the group consisting of Ig alpha-1 chain C region, Prothrombin, Apolipoprotein D, Uromodulin, Glycophorin-C, Zinc-alpha-2-glycoprotein, Heparin sulphate proteoglycan, Phosphoinositide-3-kinase interacting protein, and Interleukin 18 binding protein inhibitory component, and
b) detecting the presence of said one or more disease-related component(s) in the cleaned diagnostic sample of step a).

61. An in vitro method for removing one or more inhibitory component(s) from a diagnostic sample comprising a biological fluid selected from the group consisting of a secreted body fluid, an excreted body fluid and cerebral spinal fluid, said method comprising the steps of:
a) to said sample, adding one or more particle(s) having conjugated to at least a part of a surface thereof one or more ligand(s), said ligand(s) having an affinity for and being capable of binding to an inhibitory component present in said sample;
b) allowing said one or more inhibitory components to bind to said particles; and
c) removing said particles from the sample,
wherein said inhibitory component(s) is/are characterized as components capable of interfering with said diagnostic sample when used in a subsequent immunoassay and are selected from the group consisting of Ig alpha-1 chain C region, Prothrombin, Apolipoprotein D, Uromodulin, Glycophorin-C, Zinc-alpha-2-glycoprotein, Heparin sulphate proteoglycan, Phosphoinositide-3-kinase interacting protein, and Interleukin 18 binding protein inhibitory component.

62. A solid phase for use in the removal of inhibitory components from a diagnostic sample before subsequent use of said diagnostic sample in an immunoassay, said solid phase having at least two different types of ligands conjugated to at least a part thereof, said ligands having an affinity for and being capable of binding to different inhibitory components, wherein said inhibitory components are characterized as capable of interfering with said diagnostic immunoassay and selected from the group consisting of Ig alpha-1 chain C region, Prothrombin, Apolipoprotein D, Uromodulin, Glycophorin-C, Zinc-alpha-2-glycoprotein, Heparin sulphate proteoglycan, Phosphoinositide-3-kinase interacting protein, and Interleukin 18 binding protein inhibitory component.

63. A kit of parts comprising:
a) means for capturing and detecting one or more disease-related components in an immunoassay, and
one or more solid phases according to claim 62 for decreasing the amount of one or more inhibitory components in a biological fluid.
